(19) United States
(12) Patent Application Publication
(10) Pub. No.: US 2015/0243869 A1
(43) Pub. Date: Aug. 27, 2015

(54) SELF DOPING MATERIALS AND METHODS

(71) Applicant: The Regents of the University of California, Oakland, CA (US)

(72) Inventors: Rachel Segalman, Santa Barbara, CA (US); Boris Russ, Santa Barbara, CA (US); Fulvio Brunetti, Santa Barbara, CA (US); Craig Hawker, Santa Barbara, CA (US); Michael L. Chabinyc, Santa Barbara, CA (US); Jeffrey J. Urban, Santa Barbara, CA (US)

(21) Appl. No.: 14/626,695

(22) Filed: Feb. 19, 2015

Related U.S. Application Data
(60) Provisional application No. 61/942,511, filed on Feb. 20, 2014.

(51) Int. Cl. H01L 35/24; C07D 471/06

(52) U.S. Cl. CPC H01L 35/24; C07D 471/06

(57) ABSTRACT

A an organic material is shown including a conjugated core, one or more electron donating moieties, and a non-conjugated spacer coupled between the conjugated core and the electron donating moiety. Methods of forming the organic material include solution based processing. One example of an organic material includes a self-doping n-type organic material.

### CLAIM OF PRIORITY

This application claims the benefit of priority under 35 U.S.C. §119(e) of U.S. Provisional Patent Application Ser. No. 61/942,511, entitled “SELF-DOPING MATERIALS FOR N-TYPE ORGANIC THERMOELECTRIC APPLICATIONS,” filed on Feb. 20, 2014, the benefit of priority of each of which is claimed hereby, and which is incorporated by reference herein in its entirety.

### STATEMENT REGARDING FEDERALLY SPONSORED RESEARCH OR DEVELOPMENT

This invention was made with Government support under FA9550-12-1-0002 awarded by the Air Force Office of Sponsored Research (AFOSR). The Government has certain rights in the invention.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### TECHNICAL FIELD

This invention relates to doped organic material and methods. In one example, this invention relates to organic thermoelectric materials and devices, and their associated methods.

### BACKGROUND

Building efficient organic thermoelectric architectures may require complementary p-type (hole transporting) and n-type (electron transporting) components. While several high performance hole-transporting polymers have been developed, the design of n-type organics has proven challenging, and thermoelectric studies of organic n-type systems are scarce.

### BRIEF DESCRIPTION OF THE DRAWINGS

FIG. 1 shows a schematic diagram of a self-doping molecule according to an example of the invention.

FIG. 2 shows experimental electrical conductivity data for a self-doping material according to an example of the invention.

FIG. 3 shows experimental power factor data for a self-doping material according to an example of the invention.

FIG. 4 shows experimental optical absorption data for a self-doping material according to an example of the invention.

FIG. 5 shows experimental paramagnetic resonance data for a self-doping material according to an example of the invention.

FIG. 6 shows experimental electrical conductivity data for a self-doping material according to an example of the invention.

FIG. 7 shows example chemical structures for a self-doping molecule to an example of the invention.

FIG. 8 shows experimental electrical conductivity data for a self-doping material according to an example of the invention.

FIG. 9 shows experimental power factor data for a self-doping molecule material to an example of the invention.

FIG. 10A shows experimental attenuation coefficient data for a self-doping material according to an example of the invention.

FIG. 10B shows experimental paramagnetic resonance data for a self-doping material according to an example of the invention.

FIG. 11 shows micrographs of different film morphologies in self-doping materials according to an example of the invention.

FIG. 12A shows one example chemical structures for a self-doping molecule to an example of the invention.

FIG. 12B shows another example chemical structures for a self-doping molecule to an example of the invention.

FIG. 12C shows another example chemical structures for a self-doping molecule to an example of the invention.

FIG. 12D shows another example chemical structures for a self-doping molecule to an example of the invention.

FIG. 12E shows another example chemical structures for a self-doping molecule to an example of the invention.

FIG. 12F shows another example chemical structures for a self-doping molecule to an example of the invention.

FIG. 13 shows experimental paramagnetic resonance data for a self-doping material according to an example of the invention.

FIG. 14 shows atomic force micrographs for self-doping materials according to an example of the invention.

FIG. 15 shows a method of forming a material according to an example of the invention.

FIG. 16A shows a block diagram of a doped organic electronic device using at least one self-doping material according to an example of the invention.

FIG. 16B shows another block diagram of a doped organic electronic device using at least one self-doping material according to an example of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

The invention is described in additional detail in the following pages. All cited references are incorporated herein by reference in their entirety.

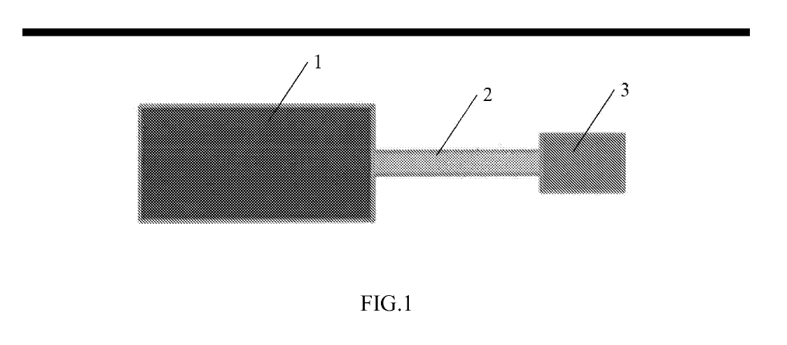

This disclosure includes the use of self-doping organic materials. In selected examples, this disclosure includes the use of self-doping organic materials for thermoelectric applications. In FIG. 1, one example of an organic material addressed by this disclosure is shown. A conjugated core (element 1) connected to anon-conjuated spacer(s) (element 2), which tether(s) an electron donating moiety (moieties) (element 3). Self-doping refers to the ability of the tethered electron donating unit to transfer charge to the conjugated molecular core.

(1) The acceptor core (element 1) can be a small molecule or polymer containing a conjugated core, including, but not limited to, perylene diimide backbones.

(2) Non-conjugated spacers (element 2) may be attached either at the bay positions of the backbone or the N,N terminal positions. Embodiments of spacers may include chains containing methylene units, pegylated components, and polyimide-based units.

(3) An even or odd number of non-conjugated spacers (element 2) may be attached. In the case where an even number is attached, these may be symmetric or asymmetric in nature.

(4) The molecular doping elements (electron donating moieties) (element 3) can either induce charge transfer in their original molecular state or after transformation by an external stimulus, such as heat, humidity, light, pH, etc.

(5) One embodiment of these molecular doping elements (element 3) includes incorporation of charged amine based end groups in the presence of appropriate counterion, such as iodine, chlorine, bromine, fluorine, hydroxide, PH6-, BF4-, CN—, etc.

(6) The self-doping molecular design strategy can be coupled with external doping strategies for additional tuning of thermoelectric properties. The molecular dopant can include hydrazine, sodium borohydride, N-DMBI derivatives, etc.

(7) Thin films of the materials addressed by this disclosure have electrical conductivities at least 0.001 S/cm.

### Thermoelectric Application

On example use of materials described in the present disclosure includes thermoelectric applications. Organic thermoelectrics have attracted considerable research interest, propelled by the promise of realizing flexible, large-area, and low-cost modules. Building efficient thermoelectric architectures capable of achieving this overarching goal requires high performance complementary p-type (hole transporting) and n-type (electron transporting) materials. Although the thermoelectric performance of p-type organic materials is rapidly advancing, the performance of n-type organic electronic materials has not benefited from the same level of innovation. Finding stable n-type doping strategies has been difficult because organic semiconductors generally have small electron affinities (−3 to −4 eV). Consequently, very few materials have been identified as n-type organic thermoelectrics. Of these materials, vapor doped fullerenes and powder-processed organometallic poly(Ni 1,1,2,2-ethenetetrathiolate) derivatives have shown the highest n-type thermoelectric performance, with electrical conductivities as high as 9 S/cm and 40 S/cm and power factors up to 30 μWm⁻¹K⁻² and 70 μWm⁻¹K⁻², respectively. However, these materials are not amenable to solution-processing. Record thermoelectric performance for solution-processed organic thermoelectric materials was recently reported by Schlitz and coworkers, who demonstrated solution doping of a high mobility n-type polymer, poly[N,N′-bis(2-octyl-dodecyl)-1,4,5,8-napthalenedicarboximide-2,6-diyl]-alt-5,5′-(2,2′-bithiophene)] (P(NDIOD-T2), using dihydro-1H-benzolinidazol-2-yl (N-DBI) derivatives.

Example materials of the present disclosure exhibit electrical conductivities of nearly 10⁻² S/cm and power factors of 0.6 μWm⁻¹K⁻². In contrast to this promising extrinsic doping approach, the present disclosure shows thermoelectric properties of self-dopable perylene diimides (PDI), in which a charged doping group is intrinsically tethered to the conjugated backbone. It is demonstrated that self-doped PDIs have the highest n-type thermoelectric performance of solution-processed organic materials reported to date and present molecular design strategies to guide future technological innovation in this new class of thermoelectric materials.

The present disclosure includes self-doping organic materials for solution-processable thermoelectric applications. In one example, a charged doping group is tethered to the conjugated backbone. In one example, the self-doping organic material includes a class of perylene diimides (PDI), but can easily be extended to other molecular core system by one knowledgeable in the field. Self-doped materials are shown that possess the highest n-type thermoelectric performance of solution-processed organic materials reported to date. Therefore, the doping strategy and molecular design examples presented hold promise for future technological innovation in the development of thermoelectric materials.

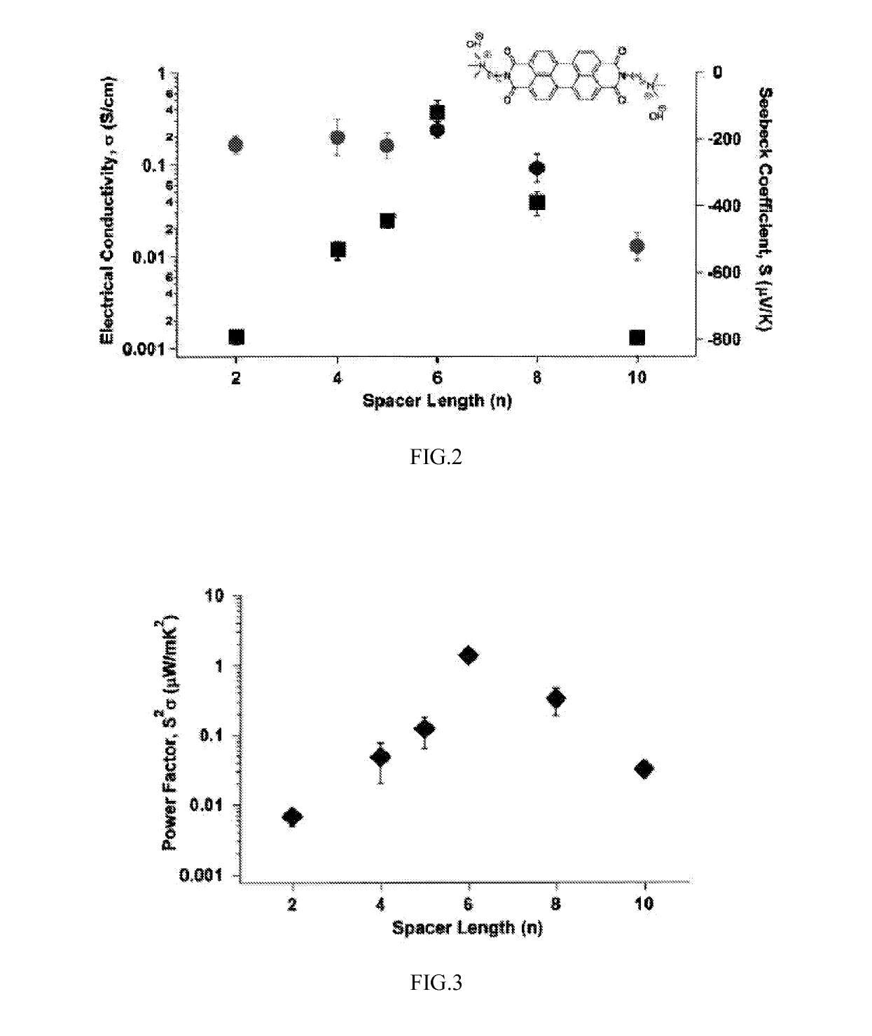

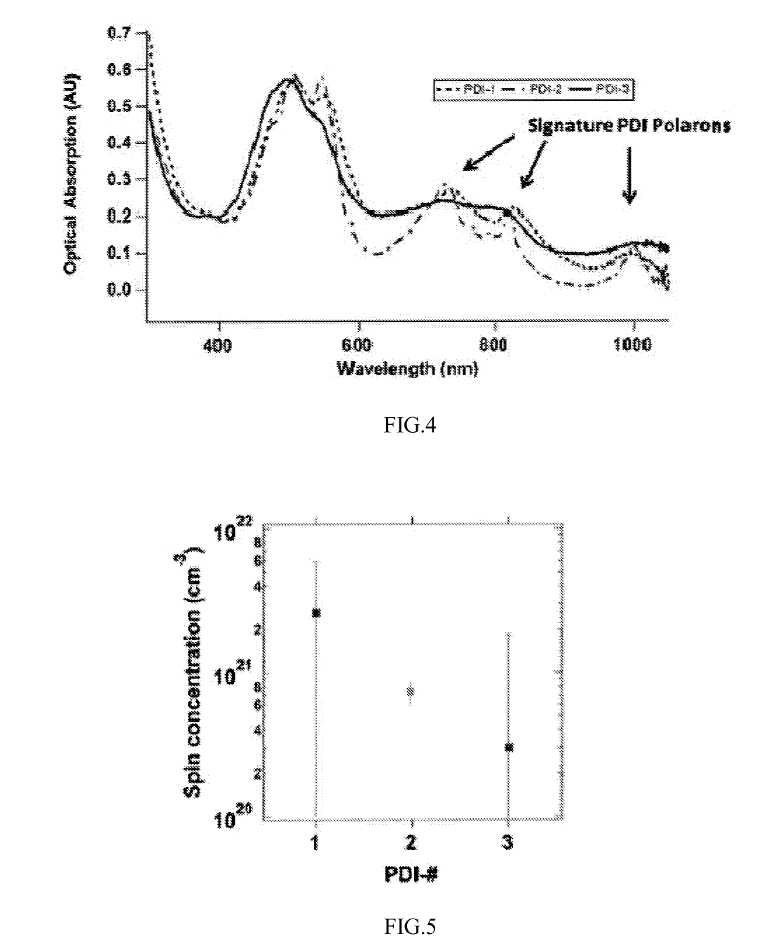

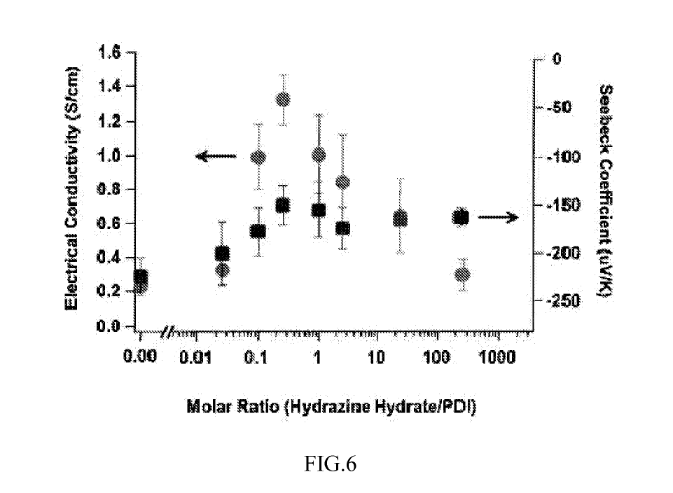

FIG. 2 shows how modification of the spacer length between the charged side groups and the perylene diimide core increases the electrical conductivity of thin films 100× to record values for a solution processed small molecule n-type material. FIG. 3 shows that the molecular design modifications also resulted in record power factors, which are one important performance metric for thermoelectric materials. FIG. 4 shows UV/VIS/NIR data that demonstrates that thermal activation induces the formation of polaron charge carriers. FIG. 5 shows electron paramagnetic resonance data. The data shows the universal ability of the doping moieties to induce high charge densities (on the order of the molecular density) in thin film materials. FIG. 6 shows electrical conductivity data of a self-doped material according to an example of the invention. The example of FIG. 6 measures a sample using hydrazine hydrate as a secondary dopant. The novel molecular design strategy in these thermoelectric materials can be coupled with other external doping strategies for additional tuning of thermoelectric properties.

PDIs have been widely examined for their n-type behavior due to their ease of synthesis, low cost, and optical and electrical transport properties that can be broadly tuned through controlled chemical transformations. The low lying LUMO (near −4.0 eV) enables electron injection from a variety of metals and provides electrochemical stability. Furthermore, water soluble derivatives can be accessed through simple chemical modifications of the PDI core, enabling a pathway toward green, solution processing of thin films.

Molecular functionalization also has potential for enabling localized dopants in assembled thin films. It has been demonstrated that in addition to providing aqueous solubility, charged ethyl side chains can facilitate a controllable and reversible self-doping mechanism activated through a low temperature thermal treatment of cast films. Capitalizing on this self-doping phenomenon, conductivities as high as 10⁻³ S/cm are shown, and showcase the material's application as an effective hole-blocking layer. Further n-type performance enhancements may be achieved by combining this self-doping mechanism with proper modification of the PDI side chain length, which has been shown to strongly change morphology and electronic properties of the resulting thin films. In this disclosure, it is shown that thin film electronic transport properties can be dramatically modified through molecular design of the alkyl spacer length between the charged side groups and the PDI backbone, suggesting that both morphology (which changes as a result of structural modification) as well as self-doping play a crucial role in thermoelectric performance. Physical characterization of a series of water-soluble PDIs and characterization of their electrical conductivities and thermoelectric power factor (as high as 0.5 S/cm and 1.4 μW/mK², respectively) demonstrate the attractive n-type thermoelectric properties accessible for these materials. Taking advantage of these characteristics, the highest n-type thermoelectric performance for any solution processed small molecule is shown in the present disclosure. Moreover, the synthetic variability inherent in these systems suggests promising strategies for achieving future performance enhancements.

### Synthesis Examples

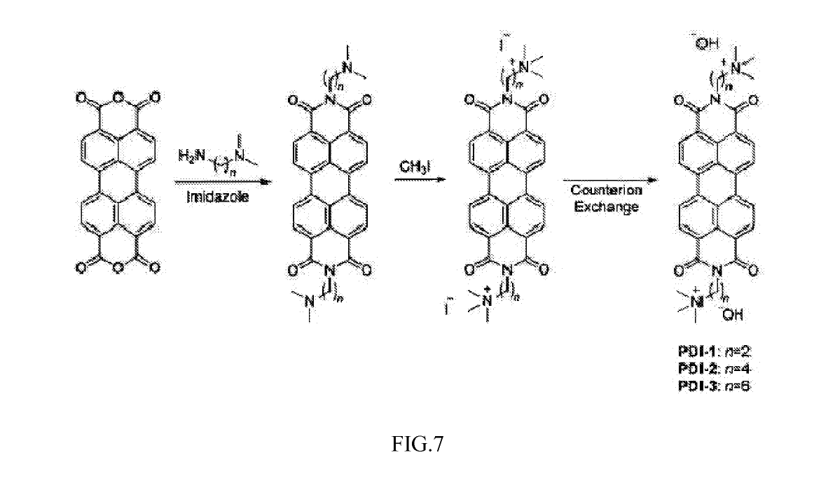

Initially, a series of functionalized perylene diimide derivatives containing two, four, and six methylene spacer groups between quaternary amine substituents and the PDI nucleus were prepared and fully characterized as shown in FIG. 7. Chemical structures of PDI small molecules with varying alkyl length (n) between the PDI backbone and the charged side group are shown in FIG. 7.

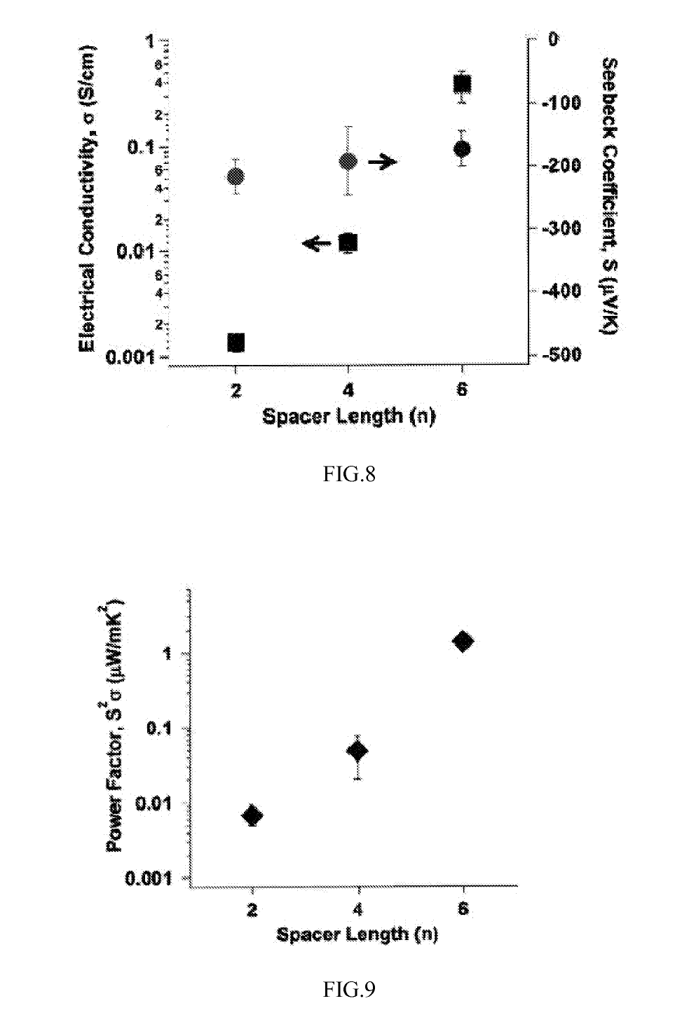

In order to characterize the thermoelectric properties of the PDI materials, thin films were dropcast onto glass substrates with pre-patterned gold contacts and thermally treated prior to property measurements. Synthetically, the nature of the charged side groups dictates interesting thermoelectric behavior as shown in FIGS. 8 and 9. FIG. 8 shows increasing the length of the alkyl chain between the charged side group and the PDI backbone from two to six methylene groups enhances electrical conductivity 100-fold. The Seebeck coefficient is negative (n-type electronic transport) and remains approximately constant with spacer length. FIG. 9 shows extending the alkyl spacer length boosts the thermoelectric power factor by two orders of magnitude.

The thermopower of all three variants is negative, confirming that n-type electrical transport is dominant. In one example, the thermopower is invariant to alkyl spacer length. Combined, the electrical conductivity and thermopower properties yield a power factor as high as 1.4 μW/mK² for films of PDI-3 (FIG. 9).

To elucidate the nature of the observed electrical conductivity (a), both the charge carrier density (n) and mobility (μ) should be considered (σ=neμ, where e is the elementary charge). Self-doping in PDI-1 increases the polaron charge carrier density, modifying the electrical conductivity many orders of magnitude. In one example, the transformation of ionic species into radical anions may be driven by molecular compaction and the associated deshielding of ions during solvent drying. Similar driving forces for generation of charge carriers have been suggested in recent findings demonstrating effective doping of fullerenes with tetrabutylammonium salts. Given the minor changes made to the side-chains, it is expected that the doping mechanism in PDI-2 and PDI-3 is similar to that in PDI-1 and also results in polaron charge carriers. To confirm the nature of the charge carriers, UV-VIS-NIR absorption spectra of all three materials were measured on thermally treated samples.

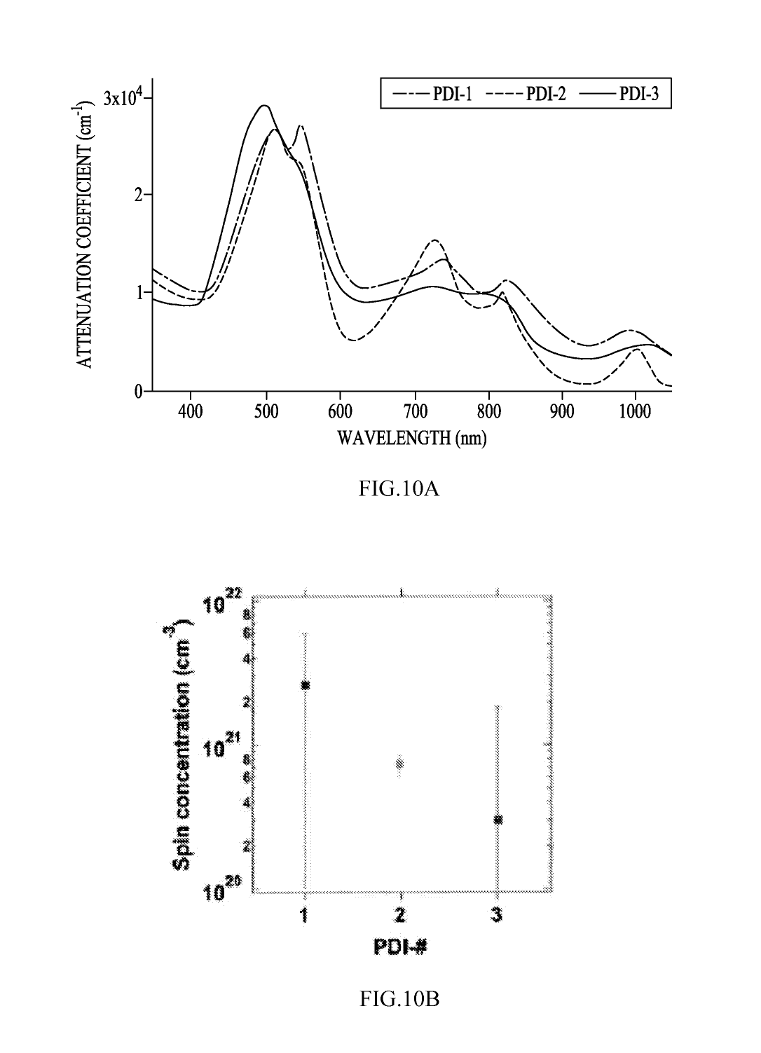

As shown in FIG. 10A, the spectra verify that all three materials have similar optical transitions in the visible range and three characteristic peaks in the visible-near infrared region (˜730 nm, 815 nm, and 1000 nm) representative of the PDI anion (polaron charge carriers). The polaron charge carrier densities were found to be within an order of magnitude (3×10²⁰-3×10²¹ polarons/cm³) for the three PDI derivatives as quantified by electron paramagnetic resonance (EPR) measurements (FIG. 10B). While these PDI variants are heavily doped (on the order of the molecular density), a population of neutral molecules is still present; the absorption in the visible region in the UV-VIS-NIR spectra is consistent with this observation.

FIG. 10A shows self-doping in all three PDI derivatives leads to high polaron charge carrier concentrations. Dropcast films (˜200 nm thick on glass) of PDI-1, PDI-2, and PDI-3 indicate similar optoelectronic transitions regardless of spacer length. Spectra of all three materials display the characteristic peaks for PDI radical anion (polaron) charge carriers (˜730 nm, 815 nm, and 1000 nm). The peak broadening seen in PDI-3 is attributed to significant intermolecular interactions. FIG. 10B shows the spin concentrations (density of polaron charge carriers) in PDI-1, PDI-2, and PDI-3 are within an order of magnitude of each other (10²⁰-10²¹ carriers/cm³) as determined by quantified EPR methods (see Supporting Information for details). These results suggest that doping level cannot explain the observed differences in electrical conductivity between the three PDI variants.

The EPR results confirm that the observed variations in electrical conductivity are not the result of differences in doping level. In fact, the charge carrier densities appear to be inversely correlated with the electrical conductivity measurements in the three variants. Therefore, we posit that alkyl spacer length influences electrical conductivity through large changes in the apparent thin film electron mobility (a combined effect of intra- and inter-domain transport properties). The molecular structure in PDI materials is known to affect film packing morphology which has been demonstrated to impact the observed carrier mobility in devices such as thin film transistors.

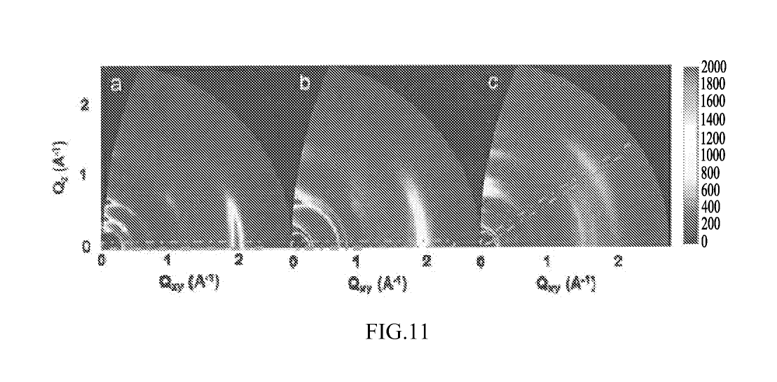

To investigate this hypothesis, we used grazing incidence wide angle X-ray scattering (GIWAXS) analysis to compare the morphologies of PDI-1, PDI-2, PDI-3 in dropcast films. The PDI films were found to be polycrystalline (FIG. 11). FIG. 11 shows that tuning the length of the alkyl spacer between the charged side groups and the PDI core leads to dramatic changes in thin film morphology. GIWAXS patterns are shown for the three PDI derivatives (a) PDI-1 (b) PDI-2 (c) PDI-3 (see Experimental Section for film preparation details). While detailed crystallographic indexing is not possible from this complex data, it is clear that the three systems have different overall structures indicating that morphology may be playing a leading role in increasing the conductivity of PDI-3 relative to PDI-1. Interestingly, PDI-1 shows the greatest structural order, indicating that molecular orientation likely does not play a positive role in this effect. Line cuts marked by the red rectangles in the three images were used to estimate π-π spacing distances in the variants.

Sharp reflections at q values of ˜1.8 Å⁻¹ for PDI-1, PDI-2, PDI-3 (spacing of 3.39 Å, 3.49 Å, and 3.33 Å in real space, respectively) are attributed to π-π stacking between PDI units, which is comparable to reported π-π spacing distances found in other PDI systems. Due to the limited number of features in the scattering analysis, definitive crystal structure assignment is difficult. Nonetheless, it is clear that the film morphologies of the three variants are indeed different.

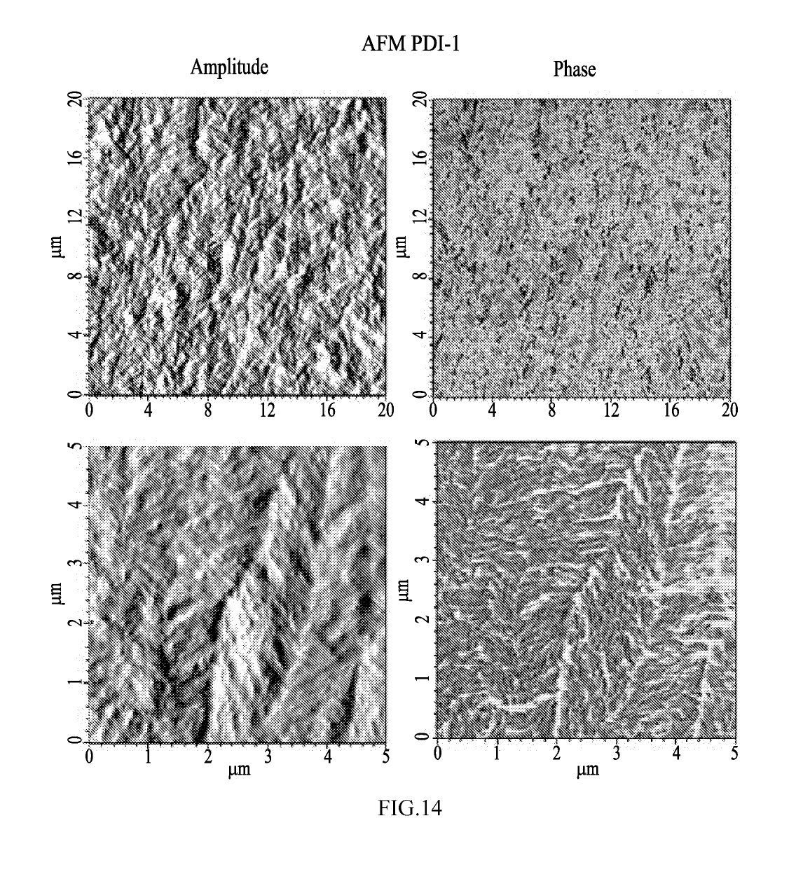

Further, applying the Scherrer formula (l=2πK/Δq, where the shape factor, K, is ˜0.9) to the observed peak broadening (Δq) in the GIWAXS data, we estimate the correlation lengths (l) in PDI-1, PDI-2, PDI-3 are 52.9 Å, 107.7 Å, and 34.1 Å, respectively. In comparison, domains in these thin films are hundreds of nanometers to microns in size, as estimated by AFM characterization (FIG. 14), suggesting that transport in these materials is most likely limited by intra-crystalline defects. Advanced processing techniques to improve the quality of crystallites (reduce defects) may help boost electronic transport properties, in addition to increasing scattering detail for better structure assignments of cast films. FIG. 14 shows AFM images of thin films of PDI-1. Both the amplitude and the phase images are shown. Domains in these thin films appear to be hundreds of nanometers to microns in size.

The observed thermopower invariance in PDI-1, PDI-2, and PDI-3 does not follow the expected inverse proportionality with conductivity and suggests an unprecedented ability to enhance the power factor (S²σ) through molecular design. This observation further supports the hypothesis that electrical conductivity is modified by morphology-induced changes in mobility, as opposed to changes in the electronic structure. Morphological changes affect scattering mechanisms that are independent of the relative position of the Fermi energy relative to the conduction band, such as boundary scattering, electron-phonon interactions, and voids. Thermoelectric transport theory suggests that such scattering events would greatly influence electrical conductivity without impact to the thermopower. This phenomena has been seen experimentally in both small molecule and polymer based systems. A deeper understanding into what specific scattering events are modified with side-chain control is the subject of ongoing investigation. Using the charge carrier density numbers from the EPR measurements, we estimate the apparent mobilities, μₐₚₚₐᵣₑₙₜ(=σ/ne), for thin films of PDI-1, PDI-2, and PDI-3 to be ˜10⁻⁶, 10⁻⁴, and 10⁻² cm²/Vs, respectively. Given that the carrier concentrations achieved in these self-dopable PDIs are high, boosting conductivity through morphology driven improvements in thin film mobility may be the most impactful strategy for further thermoelectric performance enhancement.

As shown in the present disclosure, a coupling molecular design of a perylene diimide platform with an effective doping mechanism enables a broad new direction for n-type organic thermoelectric research. It is shown that record electronic properties can be achieved by changing the alkyl spacer length between the charged side groups and the PDI backbone. The vast possibilities for enhancing the performance of water soluble n-type perylene diimide small molecules reinforce the immense potential for this system to advance the development of all solution-processed flexible thermoelectric devices.

### Experimental

PDI Synthesis.

Detailed synthetic schemes for the three materials can be found in the SI. Synthesized PDIs with iodine counterion were dissolved in DI water at concentrations of ˜2-3 mg/ml and slowly eluted through a counterion exchange column (DOWEX 550A, Sigma). The resulting solutions were all deep purple in color and were used without further treatment.

Film Preparation.

Films were dropcast on glass substrates (1 cm×1 cm, 1 mm thick). The substrates were pre-cleaned by sonicating in successive baths: 1% aqueous Hellmanex soap solution (Sigma), water, acetone, and isopropyl alcohol for 10 minutes. Four gold electrical measurement contact pads (100 nm thick overtop 5 nm of chromium adhesion layer) were then deposited near the corners using a shadow mask in a thermal evaporator. After gold deposition, the substrate cleaning sequence was repeated and additionally the washed substrates were treated with O₂-plasma (Harrick Plasma Cleaner PDC-32G) for 15 minutes to improve wettability immediately before film deposition. Films were dropcast (70 μl, 1-3 mg/ml) at 75° C. at concentrations on a leveled hotplate and kept covered to control evaporation. Cast films were approximately 400-500 nm thick as characterized by a profilometer (Dektak 150). Films were brought into a glovebox and baked inside on a hotplate for 20 minutes at 120° C. before measurement.

Electrical Transport Measurements.

Electrical measurements were obtained as previously reported. Specifically, in-plane electrical conductivity was acquired using the four-point van der Pauw technique. Tungsten tipped micromanipulators were used to puncture through the films and make electrical contact to the Au bottom contact pads. The thermopower was measured by suspending the substrate between two temperature-controlled Peltier stages (separated ˜3 mm) and applying a small temperature difference between the stages (−20° C. to +20° C.). Thermal grease was used to ensure good thermal contact. The thermoelectric voltage was measured between two Au contact pads on separate stages using micromanipulators with tungsten probe tips. Simultaneously, the temperature of each pad was measured with a (K-type) thermocouple near the probe tip. The thermoelectric voltage versus temperature difference was always linear, yielding a negative thermopower (e.g., S=−dV/dT) for all samples. Three to five identical samples from the same solution were prepared to capture the reported sample-to-sample variations of these materials.

Other Characterization.

Thin film absorption was measured using a Cary 50 UV-VIS-NIR spectrometer. A specially built holder for the UV-VIS-NIR setup was connected to a heating element and was used to bake the thin film samples at 120° C. The measurement chamber was kept under inert gas (nitrogen) to simulate the same conditions as during the electrical measurements. EPR was performed on a Bruker EMX with samples being cast on quartz substrates and treated identically to electrical measurement samples inside a glovebox. Details of EPR sample preparation and charge quantification methodology can be found in the supplemental materials. GIWAXS was performed at Stanford Synchrotron Radiation Lightsource beamline 11-3 using a photon energy of 12.7 keV, a MAR345 image plate area detector, and a helium filled sample chamber. The incidence angle was optimized to increase scattering from the film and minimize substrate scattering. For GIWAXS measurements films were dropcast on Si wafers from solutions identical to those used for electrical measurements and thermally treated at 120° C. for 20 min inside the sample chamber before measurement.

Materials.

All reagents from commercial sources were used without further purification. Perylene-3,4,9,10-tetracarboxylic dianhydride, imidazole, iodomethane, and 2-(dimethylamino)ethylamine were purchased from Aldrich. 4-(dimethylamino)butylamine and 6-(dimethylamino)hexylamine were purchased from Matrix Scientific. Deuterated solvents were obtained from Cambridge Isotope Laboratories, Inc.

Instrumentation.

¹H and ¹³C NMR spectra were recorded using a Varian 500 or 600 MHz spectrometer with the solvent signal as internal reference. Mass spectrometry was performed on a Micromass QTOF2 quadrupole/time-of-flight tandem mass spectrometer (ESI) or a Waters GCT Premier time-of-flight mass spectrometer (FD).

General Procedure for the Synthesis of Dimethylamino Perylene Diimides.

Perylene-3,4,9,10-tetracarboxylic dianhydride and the alkyl amine (4 eq.) were combined with imidazole in a round bottom flask equipped with a stir bar and sealed with a septum. The reaction vessel was purged with argon and subsequently heated with stirring at 130° C. for the indicated amount of time. At the conclusion of the reaction, the vessel was allowed to cool to room temperature, the contents were suspended in methanol, and the solid was collected by filtration using a 0.8 μm nylon membrane. The solid was washed with methanol and dried under vacuum to afford the pure product.

General Procedure for the Quaternization of Dimethylamino Perylene Diimides.

The dimethylamino substituted perylene diimide and iodomethane (4 eq.) were dissolved in chloroform in a round bottom flask equipped with a stir bar and condenser and refluxed for the indicated amount of time. At the conclusion of the reaction, the mixture was cooled to room temperature and filtered through a 0.8 μm nylon membrane. The solid was washed consecutively with chloroform, diethyl ether, hexane, and ethanol and dried under vacuum to afford the pure product.

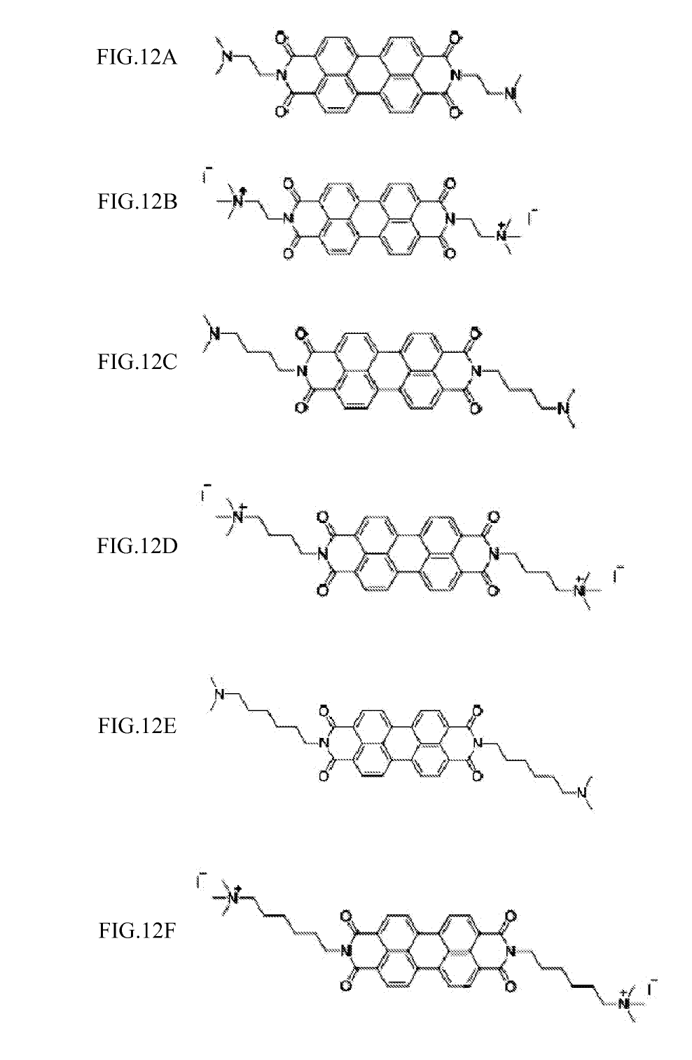

FIG. 12A shows Bis(2-(dimethylamino)ethyl)perylene diimide. The general procedure was employed using perylene-3,4,9,10-tetracarboxylic dianhydride (1.082 g, 2.757 mmol), 2-(dimethylamino)ethylamine (0.9869 g, 11.20 mmol), and imidazole (14.1 g, 207 mmol) in a 50 mL round bottom flask. After 2 h at 130° C. and workup, 1.46 g (99%) of the desired product was obtained. ¹H NMR (600 MHz, CDCl₃): δ 2.37 (s, 12H), 2.70 (t, J=7.0 Hz, 4H), 4.37 (t, J=7.0 Hz, 4H), 8.62 (d, J=8.0 Hz, 4H), 8.69 (d, J=7.9 Hz, 4H). ¹³C NMR (125 MHz, CDCl₃): δ 38.31, 45.69, 56.97, 123.28, 123.46, 126.63, 129.61, 131.66, 134.83, and 163.64. MS (FD): m/z [M]⁺ calcd for [C₃₂H₂₈N₄O₄]⁺, 532.2; found, 532.2.

FIG. 12B shows Bis(2-(trimethylammonium)ethyl)perylene diimide diiodide. The general procedure was employed using bis(2-dimethylamino)ethyl-perylene diimide (0.9048 g, 1.699 mmol), iodomethane (0.42 mL, 6.8 mmol), and chloroform (50 mL). After refluxing for 3 h and workup, 1.36 g (98%) of the desired product was obtained. ¹H NMR (600 MHz, DMSO-d6): δ 3.25 (s, 18H), 3.67 (t, J=7.4 Hz, 4H), 4.51 (t, J=7.0 Hz, 4H), 8.61 (d, J=8.1 Hz, 4H), 8.96 (d, J=8.1 Hz, 4H). ¹³C NMR (150 MHz, DMSO-d₆): δ 33.70, 52.48, 61.78, 122.41, 124.79, 125.33, 128.35, 131.00, 133.92, and 162.68. MS (ESI): m/z [M+Na]⁺ calculated for [C₃₄H₃₄I₂N₄O₄+Na]⁺, 839.0567; found, 839.0552.

FIG. 12C shows Bis(4-(dimethylamino)butyl)perylene diimide. The general procedure was employed using perylene-3,4,9,10-tetracarboxylic dianhydride (1.008 g, 2.569 mmol), 4-(dimethylamino)butylamine (1.203 g, 10.35 mmol), and imidazole (14.4 g, 211 mmol) in a 50 mL round bottom flask. After 1.25 h at 130° C. and workup, 1.44 g (95%) of the desired product was obtained. ¹H NMR (600 MHz, CDCl₃): δ 1.59-1.68 (m, 4H), 1.75-1.84 (m, 4H), 2.24 (s, 12H), 2.35 (t, J=7.6 Hz, 4H), 4.23 (t, J=7.7 Hz, 4H), 8.49 (d, J=8.0 Hz, 4H), 8.59 (d, J=7.9 Hz, 4H). ¹³C NMR (125 MHz, CDCl₃): δ 25.28, 26.19, 40.57, 45.53, 59.49, 123.17, 123.36, 126.43, 129.38, 131.45, 134.62, and 163.42. MS (FD): m/z [M]⁺ calcd for [C₃₆H₃₆N₄O₄]⁺, 588.3; found, 588.3.

FIG. 12D shows Bis(4-(trimethylammonium)butyl)perylene diimide diiodide. The general procedure was employed using bis(4-dimethylamino)butyl-perylene diimide (0.7813 g, 1.327 mmol), iodomethane (0.35 mL, 5.6 mmol), and chloroform (40 mL). After refluxing for 2 h and workup, 1.14 g (98%) of the desired product was obtained. ¹H NMR (600 MHz, DMSO-d6): δ 1.71 (p, J=7.3 Hz, 4H), 1.79-1.88 (m, 4H), 3.08 (s, 18H), 3.36-3.42 (m, 4H), 4.12 (t, J=7.4 Hz, 4H), 8.43 (d, J=8.0 Hz, 4H), 8.74 (d, J=7.9 Hz, 4H). ¹³C NMR (150 MHz, DMSO-d₆): δ 19.87, 24.44, 52.35, 64.98, 122.73, 124.21, 125.42, 128.30, 130.95, 133.76, and 162.77. MS (ESI): m/z [M+Na]⁺ calcd for [C₃₈H₄₂I₂N₄O₄+Na]⁺, 895.1193; found, 895.1182.

FIG. 12E shows Bis(6-(dimethylamino)hexyl)perylene diimide. The general procedure was employed using perylene-3,4,9,10-tetracarboxylic dianhydride (1.042 g, 2.656 mmol), 6-(dimethylamino)hexylamine (1.522 g, 10.55 mmol), and imidazole (17.1 g, 251 mmol) in a 50 mL round bottom flask. After 1.5 h at 130° C. and workup, 1.64 g (96%) of the desired product was obtained. ¹H NMR (600 MHz, CDCl₃): δ 1.37-1.55 (m, 12H), 1.77 (p, J=7.8 Hz, 4H), 2.21 (s, 12H), 2.26 (t, J=7.5 Hz, 4H), 4.18 (t, J=7.4 Hz, 4H), 8.41 (d, J=8.0 Hz, 4H), 8.54 (d, J=7.9 Hz, 4H). ¹³C NMR (125 MHz, CDCl₃): δ 27.30, 27.40, 27.83, 28.12, 40.71, 45.62, 59.97, 122.48, 122.96, 125.40, 128.64, 130.64, 133.52, and 162.76. MS (FD): m/z [M]⁺ calcd for [C₄₀H₄₄N₄O₄]⁺, 644.3; found, 644.3.

FIG. 12F shows Bis(6-(trimethylammonium)hexyl)perylene diimide diiodide. The general procedure was employed using bis(6-dimethylamino)hexyl-perylene diimide (0.9086 g, 1.409 mmol), iodomethane (0.35 mL, 5.6 mmol), and chloroform (50 mL). After refluxing for 2 h and workup, 1.28 g (98%) of the desired product was obtained. ¹H NMR (600 MHz, DMSO-d₆): δ 1.36 (p, J=7.2 Hz, 4H), 1.45 (p, J=7.2 Hz, 4H), 1.67-1.75 (m, 8H), 3.04 (s, 18H), 3.26-3.31 (m, 4H), 4.08 (t, J=7.5 Hz, 4H), 8.50 (d, J=7.9 Hz, 4H), 8.83 (d, J=7.9 Hz, 4H). ¹³C NMR (125 MHz, DMSO-d6): δ 22.08, 25.68, 26.18, 27.32, 52.14, 65.28, 121.75, 123.62, 124.46, 127.54, 130.19, 133.04, and 162.23. MS (ESI): m/z [M-I]⁺ calcd for [C₄₂H₅₀IN₄O₄]⁺, 801.2871; found, 801.2861.

### Electron Paramagnetic Resonance (EPR) Analysis

EPR samples of ethyl, butyl, and hexyl PDI were prepared by dropcasting solutions of each onto 2 cm by 2 cm quartz microscope cover slides. Each solution was approximately 1 mg/mL solution of PDI/deionized water. These samples were dried at 75° C. for 45 minutes. Upon drying, they were transferred into a dry glovebox with a nitrogen atmosphere. Inside the glovebox, the samples were cleaved into approximately 3 mm-wide pieces; the most uniform and rectangular pieces were saved as the final samples. Before measurement, samples were baked for 20 minutes at 120° C. inside the glovebox and then inserted into 4 mm-diameter quartz EPR tubes. These tubes were sealed with plastic caps and Teflon tape inside the glovebox. They were then transferred outside of the glovebox and their EPR spectra were measured within two hours.

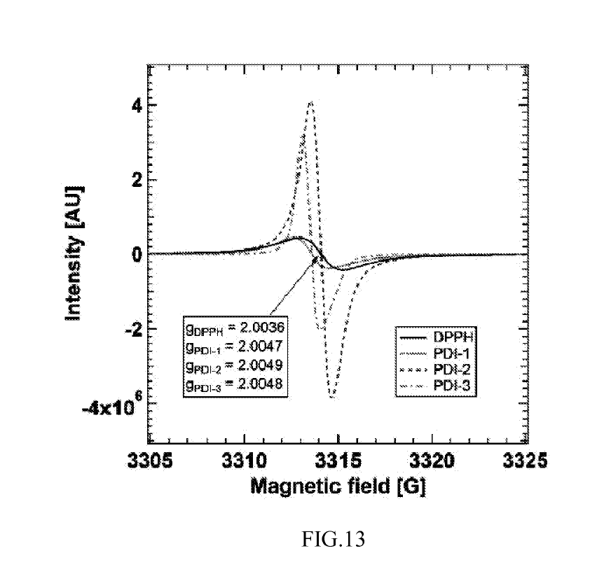

Spin concentrations were determined by comparing the integrated signal intensity of a sample with the integrated signal intensity of a standard of known concentration. In this experiment, the spin concentration calculations were complicated by the variation in film thickness that resulted from the dropcasting. The thickness of each sample was measured using profilometry. The film was scraped off in three locations down to the substrate and the average difference in thickness across these sites was quoted as the average thickness of the dropcasted film. To calculate the spin concentrations, we measured the samples after a 20 minute, 120 C bake and compared the normalized, integrated intensity of the samples to the normalized, integrated intensity of a standard material. 2,2-diphenyl-1-picrylhydrazyl (DPPH) was used as the standard sample and a calibrator for determining the g-factor of each material. Error in comparing the spin concentration stemmed from uncertainty with regards to the concentration of the standard and the error in measuring the length, width, and thickness of each sample. Raw EPR signatures are shown in FIG. 13. Specifically, FIG. 13 shows EPR signatures for PDI-1, PDI-2, and PDI-3 in comparison to a reference sample of DPPH.

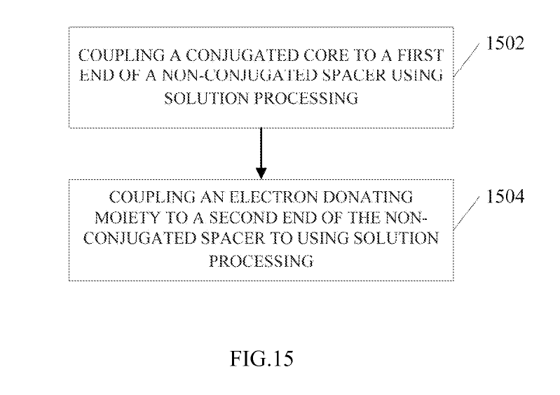

FIG. 15 shows a method of forming an organic material. In operation 1502, a conjugated core is coupled to a first end of a non-conjugated spacer using solution processing. In operation 1504, an electron donating moiety is coupled to a second end of the non-conjugated spacer to using solution processing. Although FIG. 15 shows one order of operation, the steps 1504 and 1502 may also be reversed, The conjugated core and the electron donating moiety can be coupled to the non-conjugated spacer in any order. Examples of solution processing include, but are not limited to, methods described in relation to FIGS. 12A-12F.

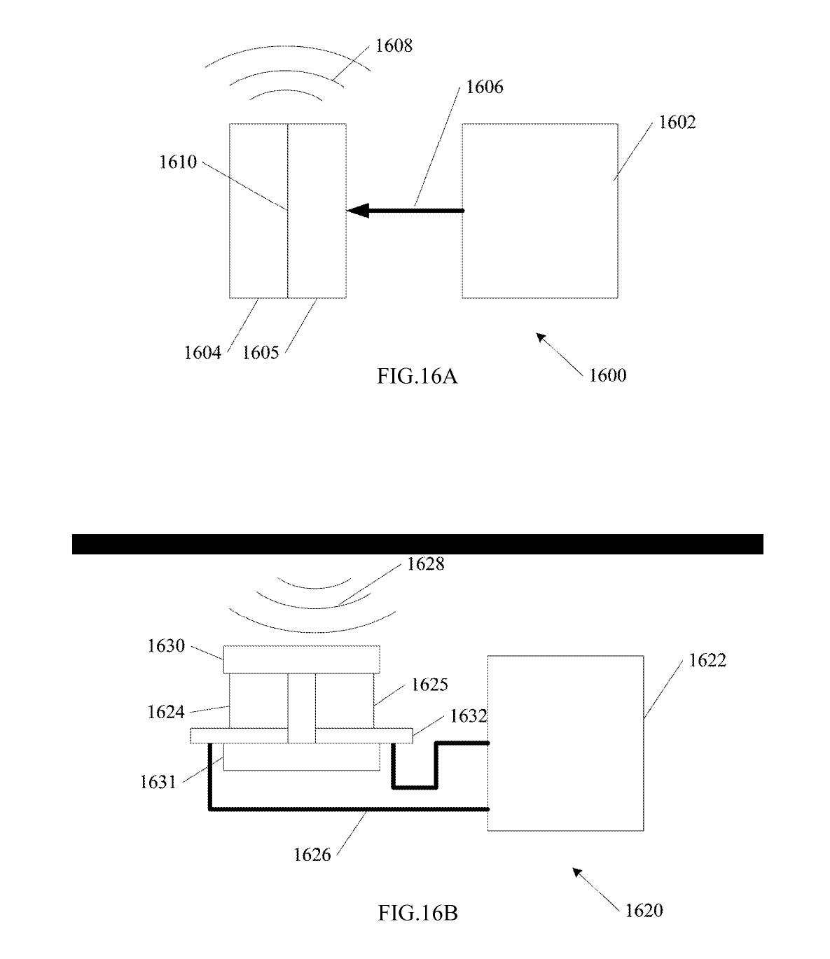

FIG. 16A shows one example of a doped organic electronic device 1600 according to an embodiment of the invention. In one example, the doped organic electronic device 1600 includes a thermoelectric device. The doped organic electronic device 1600 includes an organic p-type doped component 1604, and a self-doping organic n-type component 1605 coupled to the organic p-type doped component 1604 to form a p-n junction 1610. Electrical circuitry 1606 is coupled to the p-n junction 1610. In the example of FIG. 16A, a source of electricity 1602 is coupled to the electrical circuitry 1606, and is used to induce heat or cold, as indicated by lines 1608, when electricity in provided.

FIG. 16B shows another example of a doped organic electronic device 1620 according to an embodiment of the invention. In one example, the doped organic electronic device 1620 includes a thermoelectric device. The doped organic electronic device 1620 includes an organic p-type doped component 1624, and a self-doping organic n-type component 1625 coupled to the organic p-type doped component 1624 electrically in series, and thermally in parallel. In the example shown, a first thermal component 1630 is shown, and a second thermal component 1631, wherein the first and second thermal components have a temperature differential. In one example, the first thermal component 1630 is higher in temperature than the second thermal component 1631. In one example, the first thermal component 1630 is lower in temperature than the second thermal component 1631. Examples of p-type components include organic, inorganic, and organic/inorganic composite p-type components.

Electrical circuitry 1626 is coupled to leads 1632 that connect to the p-type doped component 1624, and the self-doping organic n-type component 1625. In the example of FIG. 16B, a change in temperature, indicated by lines 1628, is applied to the organic electronic device 1620. As a result, electricity is generated, and flows through electrical circuitry 1626 to an electric device 1622. In one example the electric device 1622 may be powered by the electrical circuitry 1626. In another example, the electric device 1622 may detect a charge from the electrical circuitry 1626.

To better illustrate the method and device disclosed herein, a non-limiting list of embodiments is provided here:

Example 1 includes an organic thermoelectric material, including a conjugated core, one or more electron donating moieties, and a non-conjugated spacer coupled to the conjugated core and to the electron donating moiety.

Example 2 includes the organic thermoelectric material of example 1, wherein the non-conjugated spacer includes 2 to 10 methylene units.

Example 3 includes the organic thermoelectric material of example 1, wherein the non-conjugated spacer includes 2 to 6 methylene units.

Example 4 includes the organic thermoelectric material of any one of examples 1-3, wherein the conjugated core comprises a perylene diimide (PDI).

Example 5 includes the organic thermoelectric material of any one of examples 1-4, wherein the non-conjugated spacer is composed of at least one unit selected from the group of units consisting of methylene units, pegylated units, and polyimide-based units.

Example 6 includes the organic thermoelectric material of any one of examples 1-5, wherein the electron donating moiety includes a charged amine based end group in the presence of an associated counter ion, wherein the counter ion is selected from a group consisting of iodine, chlorine, bromine, fluorine, hydroxide, PF6-, BF4-, and CN—.

Example 7 includes the organic thermoelectric material of any one of examples 1-6, further comprising a secondary dopant selected from the group of dopants consisting of hydrazine, sodium borohydride, and N-DMBI derivatives.

Example 8 includes a method comprising coupling a conjugated core to a first end of a non-conjugated spacer using solution processing, and coupling an electron donating moiety to a second end of the non-conjugated spacer to using solution processing.

Example 9 includes the method of example 8, wherein coupling a conjugated core to a first end of a non-conjugated spacer includes coupling the conjugated core to the first end of a methylene chain spacer.

Example 10 includes the method of any one of examples 8-9, wherein coupling a conjugated core to a first end of a non-conjugated spacer includes coupling the conjugated core to the first end of a non-conjugated spacer chosen from a group consisting of pegylated chains, and polyimide based chains.

Example 11 includes the method of any one of examples 8-10, wherein coupling a conjugated core to a first end of a non-conjugated spacer includes coupling a perylene diimide (PDI) core to the first end of the non-conjugated spacer.

Example 12 includes the method of any one of examples 8-11, wherein coupling an electron donating moiety to the second end of the non-conjugated spacer includes coupling a charged amine based end group in the presence of an associated counter ion to the second end of the non-conjugated spacer.

Example 13 includes the method of any one of examples 8-12, wherein associated counter ion is chosen from a group consisting of iodine, chlorine, bromine, fluorine, hydroxide, PF6-, BF4- and CN—.

Example 14 includes a thermoelectric device, comprising an organic p-type doped component, a self-doping organic n-type component coupled to the organic p-type doped component to form a p-n junction. The self-doping organic n-type component includes a conjugated core, one or more electron donating moieties, and a non-conjugated spacer coupled to the conjugated core and to the electron donating moiety. The thermoelectric device includes electrical circuitry coupled to the p-n junction to transmit current into or away from the p-n junction.

Example 15 includes the thermoelectric device of example 14, further including a source of electricity coupled to the circuitry and configured to induce a heating or cooling effect as a result of interaction with the p-n junction.

Example 16 includes the thermoelectric device of example 14, further including an electric device coupled to the circuitry and configured to receive electricity generated by a change in temperature at the p-n junction.

While a number of advantages of embodiments described herein are listed above, the list is not exhaustive. Other advantages of embodiments described above will be apparent to one of ordinary skill in the art, having read the present disclosure. Although specific embodiments have been illustrated and described herein, it will be appreciated by those of ordinary skill in the art that any arrangement which is calculated to achieve the same purpose may be substituted for the specific embodiment shown. This application is intended to cover any adaptations or variations of the present invention. It is to be understood that the above description is intended to be illustrative, and not restrictive. Combinations of the above embodiments, and other embodiments will be apparent to those of skill in the art upon reviewing the above description. The scope of the invention includes any other applications in which the above structures and fabrication methods are used. The scope of the invention should be determined with reference to the appended claims, along with the full scope of equivalents to which such claims are entitled.

What is claimed is:

1. An organic thermoelectric material, comprising:
a conjugated core;
one or more electron donating moieties; and
a non-conjugated spacer coupled to the conjugated core and to the electron donating moiety.

2. The organic thermoelectric material of claim 1, wherein the non-conjugated spacer includes 2 to 10 methylene units.

3. The organic thermoelectric material of claim 1, wherein the non-conjugated spacer includes 2 to 6 methylene units.

4. The organic thermoelectric material of claim 1, wherein the conjugated core comprises a perylene diimide (PDI).

5. The organic thermoelectric material of claim 1, wherein the non-conjugated spacer is composed of at least one unit selected from the group of units consisting of methylene units, pegylated units, and polyimide-based units.

6. The organic thermoelectric material of claim 1, wherein the electron donating moiety includes a charged amine based end group in the presence of an associated counter ion, wherein the counter ion is selected from a group consisting of iodine, chlorine, bromine, fluorine, hydroxide, PF6-, BF4-, and CN—.

7. The organic thermoelectric material of claim 1, further comprising a secondary dopant selected from the group of dopants consisting of hydrazine, sodium borohydride, and N-DMBI derivatives.

8. A method, comprising:
coupling a conjugated core to a first end of a non-conjugated spacer using solution processing; and
coupling an electron donating moiety to a second end of the non-conjugated spacer to using solution processing.

9. The method of claim 8, wherein coupling a conjugated core to a first end of a non-conjugated spacer includes coupling the conjugated core to the first end of a methylene chain spacer.

10. The method of claim 8, wherein coupling a conjugated core to a first end of a non-conjugated spacer includes coupling the conjugated core to the first end of a non-conjugated spacer chosen from a group consisting of pegylated chains, and polyimide based chains.

11. The method of claim 8, wherein coupling a conjugated core to a first end of a non-conjugated spacer includes coupling a perylene diimide (PDI) core to the first end of the non-conjugated spacer.

12. The method of claim 8, wherein coupling an electron donating moiety to the second end of the non-conjugated spacer includes coupling a charged amine based end group in the presence of an associated counter ion to the second end of the non-conjugated spacer.

13. The method of claim 12, wherein the associated counter ion is chosen from a group consisting of iodine, chlorine, bromine, fluorine, hydroxide, PF6-, BF4- and CN—.

14. A thermoelectric device comprising:
an organic p-type doped component;
a self-doping organic n-type component coupled to the organic p-n type doped component to form a p-n junction, the self-doping organic n-type component including:
a conjugated core;
one or more electron donating moieties;
a non-conjugated spacer coupled to the conjugated core and to the electron donating moiety; and
electrical circuitry coupled to the p-n junction to transmit current into or away from the p-n junction.

15. The thermoelectric device of claim 14, further including a source of electricity coupled to the circuitry and configured to induce a heating or cooling effect as a result of interaction with the p-n junction.

16. The thermoelectric device of claim 14, further including an electric device coupled to the circuitry and configured to receive electricity generated by a change in temperature at the p-n junction.
